(19) United States
(12) Plant Patent Application Publication
(10) Pub. No.: US 2004/0055063 P1
(43) Pub. Date: Mar. 18, 2004

(54) Tilia cordata plant named 'SHIBAMICHI GOLD'

(76) Inventor: Akira Shibamichi, Kawasguchi City (JP)

Correspondence Address: CHRISTIE, PARKER & HALE, LLP, 350 WEST COLORADO BOULEVARD, SUITE 500, PASADENA, CA, 91105, US

(21) Appl. No.: 10/246,876

(22) Filed: Sep. 18, 2002

(51) Int. Cl. A01H 5/00

(52) U.S. Cl. PLT/222

(57) ABSTRACT

A new plant variety of Tilia cordata Mill. named ‘SHIBAMICHI GOLD’ having attractive golden yellow new foliage which matures to chartreuse.

### BACKGROUND OF THE INVENTION

The present invention relates to a new and distinct variety of Tilia cordata Mill., which was developed in a controlled program in Kawasguchi City, Japan by the originator Mr. Akira Shibamichi. The varietal denomination of the new variety is ‘SHIBAMICHI GOLD’.

The genus Tilia comprises about 45 species of deciduous trees native to Eastern and Central North America; Europe; and Western Central and Eastern Asia. It is included in the family Tiliaceae which comprises about 48 genera and 725 species.

Tilia cordata is widespread and common throughout much of England, Wales, Northeast Spain, Sweden to Western Russia and Southern Caucasia. It generally grows to 30 meters tall. Leaf color is deep green depending on light intensity and cultural conditions. Leaves are generally suborbicular, abruptly acuminate, finely and scabrous serrate on the margins. Leaves are generally glossy, dark green above, glacenscent with tufts in vein axils beneath. Flower color is yellowish white, fragrant in pendulous to erect cymes.

### SUMMARY OF THE INVENTION

The new variety was discovered in a controlled breeding program of Tilia cordata and differs from its parent by having bright golden yellow new foliage which matures to chartreuse in the summer. Asexual reproduction of the new variety by grafting, has confirmed that the distinctive characteristics of the new variety are stable and transmitted to succeeding generations, and the new variety reproduces true to type.

### COMPARISON WITH PARENT

‘SHIBAMICHI GOLD’ is distinguished from its parent and all other varieties of Tila cordata of which I am aware by its bright golden yellow new foliage which matures to chartreuse.

### BRIEF DESCRIPTION OF THE ILLUSTRATIONS

The accompanying illustrations show a specimen of the new cultivar in photographic illustrations as true to color as is reasonably possible to make in illustrations of this character.

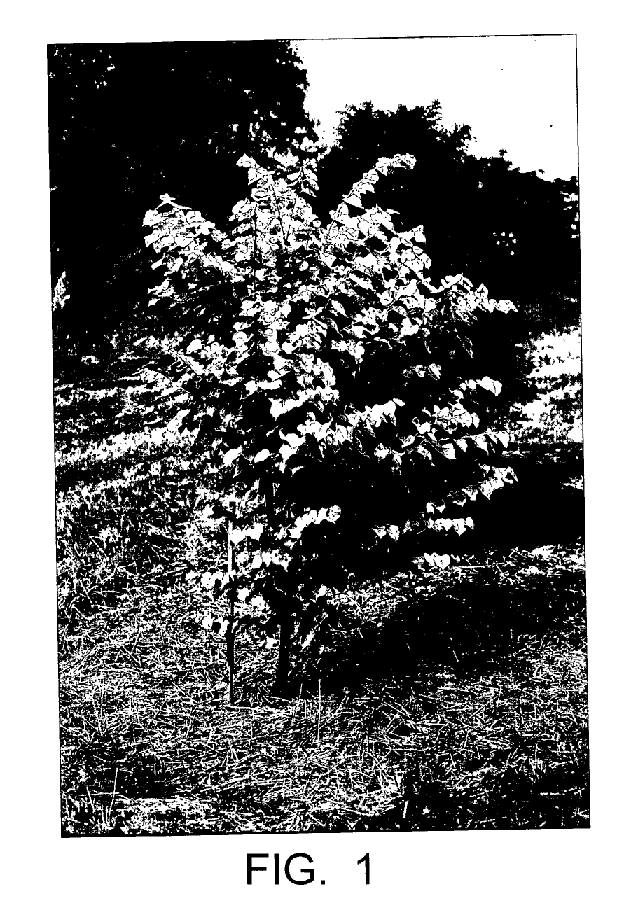

FIG. 1 shows a view of a 6 year-old specimen of ‘SHIBAMICHI GOLD’ showing the distinctive bright golden yellow new foliage and the darker chartreuse colored mature foliage.

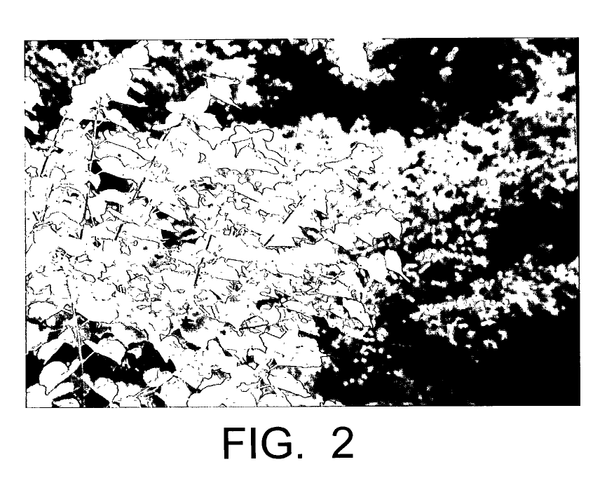

FIG. 2 shows a close-up view of the new foliage of ‘SHIBAMICHI GOLD’ showing its distinctive bright golden yellow color.

### DETAILED DESCRIPTION OF THE INVENTION

‘SHIBAMICHI GOLD’ has not been observed under all possible environmental, cultural and light conditions. The following observations and descriptions are of plants grown in York Haven, Pa. In this description, color references are to The Royal Horticultural Society Colour Chart (2001) and terminology used in the color descriptions herein refers to plate numbers in this color chart. Phenotypic expression may vary with light intensity, cultural and environmental conditions.

Classification:

Botanical.—Tilia cordata Mill. ‘SHIBAMICHI GOLD’.

Parentage.—Branch sport of Tilia cordata (unnamed, unparented).

Propagation.—Grafting.

Plant:

Size.—About 5 meters.

Age.—About 6 years old, in controlled trial conditions.

Hardiness.—USDA Zone 3 (−40° F. to −30° F.).

Vigor.—Rapid grower; approximately 0.5 meter per year.

Pests/Diseases.—None noted.

Bark.—Near Brown Group N200B Branching Typical Number: More than 1,000 lateral branches Observed Number: Approximately 100 lateral branches Size: About 1.5 meters Diameter: Up to about 8 cms Color New Shoots Upper Surface: Near Greyed-Orange Group N170A Under Surface: Near Yellow Group 151B Mature Shoots: Near Greyed-Orange Group 177C.

Leaf:

Shape.—Suborbicular to rounded cordate, apex is abruptly acuminate.

Size.—Length: Between 6.0 and 8.0 cms Width: Between 6.5 and 8.0 cms.

Arrangement.—Alternate.

Margin.—Finely and scabrous serrate.

Texture/Substance.—Pubescent.

Color.—The following color readings are of mature specimens grown in York Haven, Pa. Young Foliage Upper Surface: Near Yellow-Green Group 151A-B Under Surface: Near Yellow-Green Group 145A Mature Foliage Upper Surface: Near Green Group 143A Under Surface: Near Green Group 143C-D.

Venation.—Pattern: Upper surface, pinnate.

Petioles.—Size: About 3.5 cm long Coloration: Near Yellow-Green Group 145B Texture: Pubescent.

Internodes.—Between 2.5 and 3.7 cm.

Inflorescence: Due to the immaturity of the plant, no flowers have been produced.

I claim:

1. A new plant variety of Tilia cordata of the variety substantially as shown and described.
